(19) United States
(12) Patent Application Publication
(10) Pub. No.: US 2025/0058108 A1
(43) Pub. Date: Feb. 20, 2025

(54) ELECTRICAL STIMULATION MASSAGE DEVICE, CONTROL METHOD FOR THE DEVICE, AND STORAGE MEDIUM

(71) Applicant: SKG Health Technologies Co., Ltd., Shenzhen (CN)

(72) Inventors: Chang PENG, Shenzhen (CN); Honghong CHEN, Shenzhen (CN)

(73) Assignee: SKG Health Technologies Co., Ltd., Shenzhen (CN)

(21) Appl. No.: 18/937,723

(22) Filed: Nov. 5, 2024

Related U.S. Application Data
(63) Continuation of application No. PCT/CN2023/085757, filed on Mar. 31, 2023.

(30) Foreign Application Priority Data
May 18, 2022 (CN) 202210538975.4

(51) Int. Cl. A61N 1/36

(52) U.S. Cl. CPC A61N 1/36031

(57) ABSTRACT

An electrical stimulation massage device, a control method for the device, and a storage medium are provided. The control method includes the following. An impedance value between the electrodes arranged in pairs is detected, in response to the electrodes being adhered to the human skin and outputting an electromagnetic pulse signal. A voltage output to the electrodes is reduced in response to a determination that the impedance value decreases, when the impedance value is in a first impedance-value-range. The voltage output of the electrodes is reduced in response to a determination that the impedance value increases, when the impedance value is in a second impedance-value-range.

### CROSS-REFERENCE TO RELATED APPLICATION(S)

This application a continuation of International Application No. PCT/CN2023/085757, filed on Mar. 31, 2023, which claims priority to Chinese Patent Application No. 202210538975.4, filed May 18, 2022, the content of which are hereby incorporated by reference in their entirety.

### TECHNICAL FIELD

The disclosure relates to the field of electrical stimulation massage device, and in particular to an electrical stimulation massage device, a control method for the device, and a non-transitory storage medium.

### BACKGROUND

A massager is a non-surgical device that can help relieve pain and reduce discomfort.

In particular, a massager that uses an electrode for electric stimulation massage can adjust an output rank via buttons, remote controls, applications (APPs), etc., so as to control an output pulse of the electrode and thus control output strength with convenience. However, actual felt strength will be affected by many factors, such as the degree of adherence of the electrode, electrode material, and skin dryness. Moreover, users with different constitutions have different tolerance to current. In the medical field, there are three types of doses of electric pulse stimulation: a feeling limit, which has a limitation that the electric pulse can just trigger feeling; a contraction limit, which has a limitation that the electric pulse can just trigger muscle contraction; and a tolerance limit, which is within a limit of tolerable power.

If a present strength reaches the contraction limit, a further increase in output voltage or output current will cause the present strength to reach or exceed the tolerance limit, so that the human body will experience pain such as muscle or skin tearing.

Different degrees of adherence between the electrode and the human body and different dryness of the human skin will influence an effective contact area between the electrode and the human body. If the effective contact area is too small and an output voltage of the electrode is relatively large, an electric stimulation current output by the massager will be concentrated on part of the skin that has good adherence, so that a tingling sensation on the epidermis will be produced.

Meanwhile, during the continuous use of the massager, due to sweating or local skin heating, the skin impedance will continue to decrease or even suddenly decrease rapidly. In this case, if the output strength of the massager is still maintained at the previous state, the massage strength felt by the human body will suddenly increase, which may easily cause discomfort such as tingling or muscle spasms. Therefore, how to control the massager to output pulses of appropriate intensity under various environmental conditions is a key technology to improve the experience of the massager user.

In order to output pulses of appropriate intensity, some products in the current market, especially those with low-frequency electric stimulation output, medium-frequency electric stimulation output, and so on, use voltage amplitude/current intensity to define output ranks. The massager ranks are also set based on voltage parameters or current parameters, but this manner belongs to open-loop control. In a constant-current output mode, situations where skin impedance drops suddenly due to sweating and so on can be relieved, but it will be more painful when the contact area becomes smaller or the skin is dry. In a constant-voltage mode, the problem of experience due to the contact area between the skin and the electrode becoming smaller or due to dry skin can be relieved when the voltage is low, but there is still a risk of pain when the rank is high and the voltage output is large.

The above constant-voltage output manner and constant-current output manner cannot achieve intelligent adjustment.

### SUMMARY

In a first aspect, a control method for an electrical stimulation massage device is provided. The electrical stimulation massage device includes electrodes arranged in pairs. The electrodes are configured to be adhered to human skin at an area to be massaged. The control method includes the following. An impedance value between the electrodes arranged in pairs is detected, in response to the electrodes being adhered to the human skin and outputting an electromagnetic pulse signal. A voltage output to the electrodes is reduced in response to a determination that the impedance value decreases, when the impedance value is in a first impedance-value-range. The voltage output of the electrodes is reduced in response to a determination that the impedance value increases, when the impedance value is in a second impedance-value-range. The maximum value of the first impedance-value-range is less than or equal to the minimum value of the second impedance-value-range.

In a second aspect, an electrical stimulation massage device is provided. The electrical stimulation massage device includes a power supply, a control unit, a boosting unit, electrodes, a pulse modulation circuit, a first detecting circuit, and a second detecting circuit. The boosting unit is connected to the control unit and the power supply respectively. The boosting unit is configured to boost an input voltage of the power supply to a preset voltage under the control of the control unit, and output the preset voltage through a voltage output terminal of the boosting unit. The electrodes are configured to be adhered to human skin at an area to be massaged. An electric energy input terminal of the pulse modulation circuit is connected to the voltage output terminal of the boosting unit. A first pulse transmitting terminal of the pulse modulation circuit and a second pulse transmitting terminal of the pulse modulation circuit each are connected to an electrode. A control terminal of the pulse modulation circuit is connected to the control unit. The first detecting circuit is connected to the control unit and the voltage output terminal of the boosting unit respectively. The control unit is configured to obtain an output voltage of the boosting unit through the first detecting circuit. The second detecting circuit is connected to the control unit. A sampling resistor of the second detecting circuit is connected in series between the pulse modulation circuit and the ground. The control unit is configured to: detect an impedance value between the electrodes arranged in pairs, in response to the electrodes being adhered to human skin and outputting an electromagnetic pulse signal; reduce a voltage output to the electrodes in response to a determination that the impedance value decreases, when the impedance value is in a first impedance-value-range; and reduce the voltage output of the electrodes in response to a determination that the impedance value increases, when the impedance value is in a second impedance-value-range. The maximum value of the first impedance-value-range is less than or equal to the minimum value of the second impedance-value-range, and a voltage value of the electromagnetic pulse signal is controlled.

In a third aspect, a non-transitory computer-readable storage medium storing a computer program is provided. The computer program, when executed by a processor, is operable with the processor to implement the method in the first aspect.

### BRIEF DESCRIPTION OF THE DRAWINGS

The disclosure will be further described below with reference to the accompanying drawings and embodiments, and the accompanying drawings are as follows.

FIG. 1 is a circuit principle diagram of a pulse modulation circuit in the disclosure.

FIG. 2 is a schematic flow chart of a control method for an electrical stimulation massage device in the disclosure.

FIG. 3 is a schematic flow chart of detecting an impedance value between electrodes arranged in pairs in the disclosure.

FIG. 4 is a schematic flow chart illustrating an association between a voltage output and a present rank in the disclosure.

FIG. 5 is a schematic flow chart illustrating an association between a voltage output and a present rank in the disclosure.

FIG. 6 is a schematic flow chart illustrating entering a fourth impedance-value-range and a fifth impedance-value-range in the disclosure.

FIG. 7 is a schematic flow chart illustrating an adjustment rank and non-adjustment rank in the disclosure.

FIG. 8 is a circuit principle diagram of an electrical stimulation massage device in the disclosure.

FIG. 9 is a circuit principle diagram of a pulse modulation circuit in the disclosure.

FIG. 10 is a schematic circuit diagram of a second detecting circuit in the disclosure.

FIG. 11 is a schematic circuit structural diagram of a first detecting circuit in the disclosure.

FIG. 12 is a circuit principle diagram of a pulse modulation circuit in the disclosure.

FIG. 13 is a schematic circuit diagram of a pulse modulation circuit in the disclosure.

FIG. 14 is a circuit principle diagram of a boosting unit in the disclosure.

FIG. 15 is a schematic circuit diagram of a boosting unit in the disclosure.

### DETAILED DESCRIPTION

Embodiments of the disclosure will be described in detail with reference to accompanying drawings.

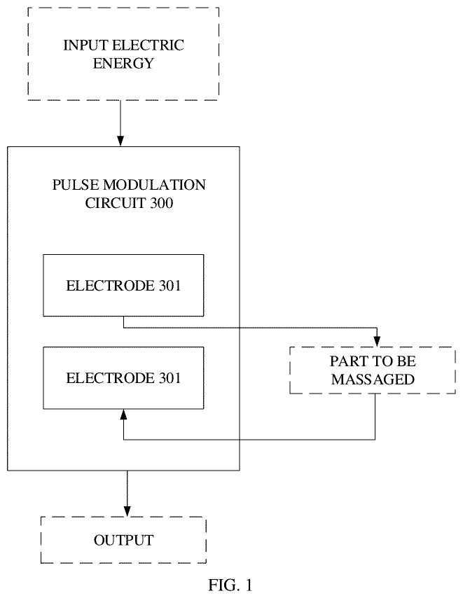

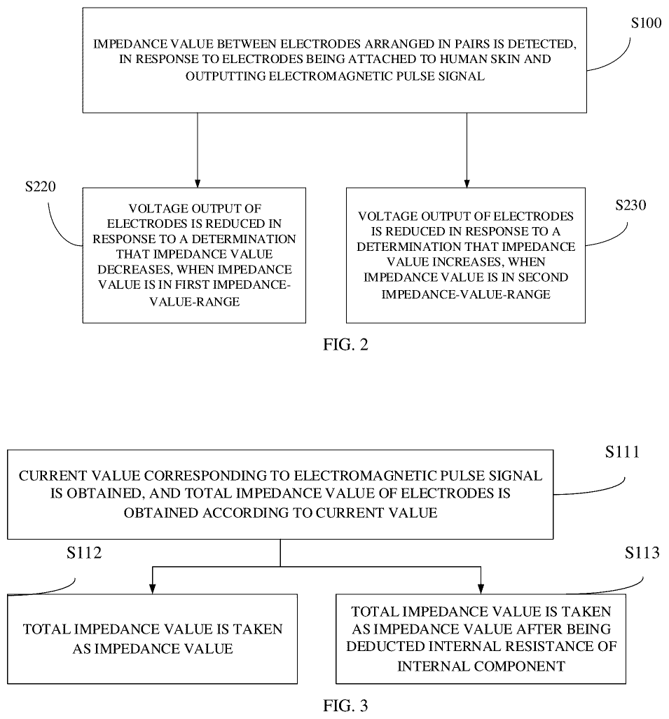

As illustrated in FIG. 1 to FIG. 3, the disclosure provides an embodiment of a control method for an electrical stimulation massage device.

For the control method for the electrical stimulation massage device, the electrical stimulation massage device includes electrodes 301 arranged in pairs which are configured to be adhered to human skin at an area to be massaged. The control method includes the following.

At S100, an impedance value between the electrodes 301 arranged in pairs is detected, in response to the electrodes being adhered to the human skin and outputting an electromagnetic pulse signal.

At S220, a voltage output to the electrodes is reduced, in response to a determination that the impedance value decreases, when the impedance value is in a first impedance-value-range.

At S230, the voltage output of the electrodes is reduced, in response to a determination that the impedance value increases, when the impedance value is in a second impedance-value-range.

The beneficial effect of the disclosure lies in that, compared with the related art, the disclosure pre-sets the first impedance-value-range and the second impedance-value-range, and adjusts the output voltage for the electrodes according to the change of the impedance value, thereby adjusting the current of the electromagnetic pulse signal output by the pulse modulation circuit, so that the human skin is prevented from being subjected to strong electric stimulation during the massage, massage without stinging is implemented, and the strength of the massage is prevented from being out of control, thereby improving the user experience.

The maximum value of the first impedance-value-range is less than or equal to the minimum value of the second impedance-value-range. The electrical stimulation massage device can be a wearable electrical stimulation massage device such as a neck massager, a waist massager, or a leg massager.

In the embodiment, the electrical stimulation massage device adjusts a voltage of an output electromagnetic pulse signal by controlling a voltage value of an input voltage. The electrodes 301 are configured to be adhered to human skin at an area to be massaged, and output the electromagnetic pulse signal, thereby generating an electric stimulation effect at a part of the human skin adhered to the electrodes 301 to perform massage.

In the operation at S100, when the electrodes 310 are adhered to the human skin and output the electromagnetic pulse signal, related electrical parameters of the electromagnetic pulse signal are obtained through a related detecting circuit, and the related electrical parameters include a value of current flowing through the human skin, a voltage value of the electromagnetic pulse signal, and an error value generated by flowing through the circuit of the electrical stimulation massage device and/or resistance generated by components and wires, so that an impedance value generated in a process of the electrodes 301 being adhered to the human skin is calculated. Moreover, the impedance value between the electrodes 301 arranged in pairs needs to be detected in real-time to perform subsequent related operations.

Referring to FIG. 3, the impedance value between the electrodes 301 arranged in pairs is detected as follows.

At S111, a current value corresponding to the electromagnetic pulse signal is obtained, and a total impedance value of the electrodes 301 is obtained according to the current value.

At S112, the total impedance value is taken as the impedance value.

Alternatively, at S113, the total impedance value is taken as the impedance value after being deducted internal resistance of an internal component.

In the operation at S111, the current value corresponding to the electromagnetic pulse signal is obtained by connecting a sampling resistor in series after the current flows through the human skin, that is, a real-time current flowing through the sampling resistor is calculated by detecting a real-time voltage of the sampling resistor. Since the voltage value of the electromagnetic pulse signal, that is, the voltage output of the electrodes 301, is known, the total impedance value of the electrodes 301 is obtained according to the current value and the voltage output. There are two judgment methods for detecting the impedance value between the electrodes 301 arranged in pairs via the total impedance value. The first is the operation at S112, which takes the total impedance value as the impedance value, and the second is the operation at S113, which takes the total impedance value as the impedance value after deducting the internal resistance of the internal component from the total impedance value. The internal resistance of the internal component mentioned above may refer to resistance of the sampling resistor, may refer to resistance of another component and wire, and of course, may also refer to a preset resistance obtained through theory.

For the operation at S220 and the operation at S230, first of all, a judgment operation, may exist between the operation at S100 and the operation at S220 or exist between the operation at S100 and the operation at S230. In the operation at S210, a judgment is made according to the impedance value detected in the above operation at S100, so as to determine whether the impedance value is in the first impedance-value-range and the second impedance-value-range. If the impedance value is in the first impedance-value-range, the operation at S220 is performed. If the impedance value is in the second impedance-value-range, the operation at S230 is performed. The first impedance-value-range and the second impedance-value-range are pre-set and are configured to reflect a range in which tingling is prone to occur, and the output voltage for the electrodes 301 needs to be adjusted according to change in the impedance value, so that the current of the electromagnetic pulse signal output by the pulse modulation circuit 100 can be adjusted, the human skin is prevented from being subjected to strong electric stimulation during the massage, and a massage without tingling is implemented.

Furthermore, when the impedance value is in the first impedance-value-range, the impedance value is kept being detected, and when the impedance value decreases, the voltage output of the electrodes 301 is reduced. Similarly, when the impedance value is in the second impedance-value-range, the impedance value is kept being detected, and when the impedance value increases, the voltage output of the electrodes 301 is reduced.

If the impedance value is in the first impedance-value-range, it means that the human skin is adhered in good condition to the electrodes 301. In this case, if the output voltage remains unchanged, when the impedance of the human body suddenly changes due to sweating or other reasons, the massage strength will also suddenly change, for example, the strength disappears, or the strength suddenly increases and the current flowing through the skin tissue exceeds a tolerance limit, leading to discomfort such as muscle cramps. Therefore, in this case, when the impedance value further decreases, the output voltage needs to be reduced to ensure that the user can experience pulse therapy with a safe and comfortable electromagnetic pulse signal voltage.

If the impedance value is in the second impedance-value-range, it means that the human skin is adhered in common condition to the electrodes 301, or means that the skin is dry, and the greater the impedance value, the worse the degree of adherence with the electrodes 301. In this case, while maintaining the massage strength, it is also necessary to prevent the output voltage from being too large and causing tingling on part of the skin. Therefore, in this case, when the impedance value further increases, the output voltage needs to be reduced to prevent the occurrence of tingling.

In this way, intelligent control and adjustment of the massage device can be achieved through the above control.

In some embodiments, the maximum value of the first impedance-value-range is less than or equal to the minimum value of the second impedance-value-range. Firstly, the first impedance-value-range and the second impedance-value-range are just two ranges where the change in the degree of adherence is slow. The two ranges can be two adjacent ranges. In this case, the maximum value of the first impedance-value-range is equal to the minimum value of the second impedance-value-range. Alternatively, a safe range may exist between the two ranges, allowing users to enjoy massage techniques with different voltage ranks in the safe range, so that the maximum value of the first impedance-value-range is less than the minimum value of the second impedance-value-range.

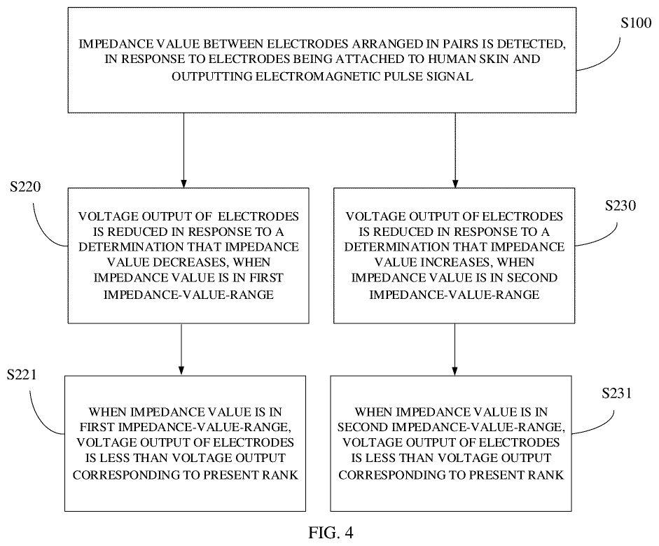

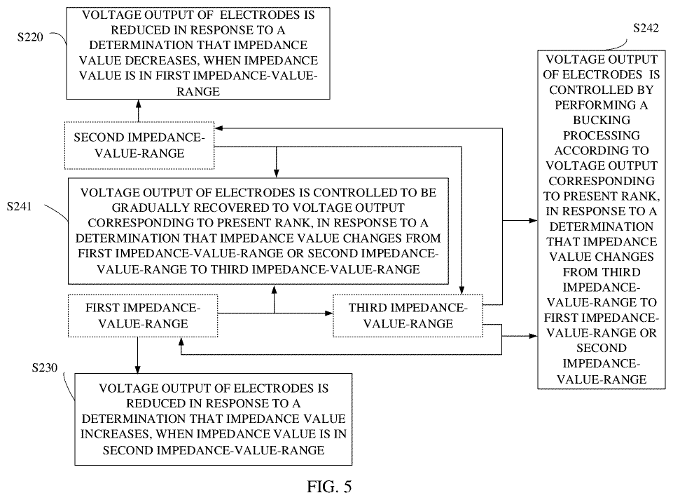

As illustrated in FIG. 4 and FIG. 5, the disclosure provides an embodiment of the association between the voltage output and the present rank.

The control method further includes the following.

At S221, when the impedance value is in the first impedance-value-range, the voltage output of the electrodes 301 is less than a voltage output corresponding to the present rank.

At S231, when the impedance value is in the second impedance-value-range, the voltage output of the electrodes 301 is less than the voltage output corresponding to the present rank.

In the embodiment, the impedance value is a safe value or is in a safe range, the voltage can be output according to a voltage of the present rank, and a massage rank is a preset voltage rank, so that different needs of different users for massage strength can be met. In addition, since the first impedance-value-range and the second impedance-value-range are just two ranges where the change in the degree of adherence is slow, directly adopting the voltage output of the present rank is likely to produce adverse effects according to the above description. Therefore, when the impedance value is in the first impedance-value-range and the second impedance-value-range, the voltage output of the electrodes 301 needs to be less than the voltage output corresponding to the present rank.

In some embodiments, a third impedance-value-range is provided. If the impedance value is in the third impedance-value-range, the voltage output corresponding to the present rank is output to the electrodes 301. The third impedance-value-range is between the first impedance-value-range and the second impedance-value-range. The third impedance-value-range can be considered as a safe range. When the impedance value is detected to be in the third impedance-value-range, it indicates that the adherence between the human skin and the electrodes 301 is normal. In this case, the voltage does not need to be adjusted, but only to meet a voltage of a rank required by the user. The voltage of the electromagnetic pulse signal is in a stable stage, and there will be no sudden change in current, no extremely small current producing no strength, and no extremely large current causing tingling.

Furthermore, in the third impedance-value-range, the impedance value will gradually change according to the user's wearing condition or skin condition. For example, after wearing for a long time, the impedance value will gradually decrease, and will slowly approach the first impedance-value-range or even enter the first impedance-value-range. If the wearing is abnormal or not in compliance with the standard, the impedance value will approach the second impedance-value-range or even enter the second impedance-value-range. Therefore, the specific control method is as follows.

At S241, the voltage output of the electrodes 301 is controlled to be gradually recovered to the voltage output corresponding to the present rank, in response to a determination that the impedance value changes from the first impedance-value-range or the second impedance-value-range to the third impedance-value-range.

At S242, the voltage output of the electrodes 301 is controlled by performing a bucking process according to the voltage output corresponding to the present rank, in response to a determination that the impedance value changes from the third impedance-value-range to the first impedance-value-range or the second impedance-value-range.

First of all, the first impedance-value-range and the second impedance-value-range are just two ranges where the change in the degree of adherence is slow. Before entering the first impedance-value-range and the second impedance-value-range, the impedance value may be in the third impedance-value-range. The impedance value is constantly changing with time or the wearing condition of the body, and will slowly enter the first impedance-value-range or the second impedance-value-range. In the third impedance-value-range, the output voltage is determined by the present rank to ensure that the user can experience massage techniques at different ranks. Therefore, when the impedance value changes from the first impedance-value-range or the second impedance-value-range to the third impedance-value-range, the voltage output of the electrodes 301 is gradually recovered to the voltage output corresponding to the present rank, and the output voltage is determined according to the present rank. In order to ensure the stability of the massage strength and maintain the massage strength favored by the user, the massage strength may not be adjusted significantly. Therefore, when the impedance value changes from the third impedance-value-range to the first impedance-value-range or the second impedance-value-range, the present rank is first obtained, and the voltage is adjusted according to the output voltage of the present rank.

In some embodiments, the voltage output of the electrodes 301 is increased, in response to a determination that the impedance value increases, when the impedance value is in the first impedance-value-range. In the entire first impedance-value-range, the voltage output of the electrodes 301 changes with the impedance value specifically as follows.

If the impedance value is in the first impedance-value-range, the voltage output of the electrodes 301 satisfies an equation

$$V 11 = a 1 + b 1 ( \frac{R 1}{X 21} ) k 1.$$

a1+b1=V12. V11 is a voltage value of the voltage output of the electrodes when the impedance value is in the first impedance-value-range. V12 is a voltage value of the present rank. a1 is a safe-voltage value. R1 is an impedance value in the first impedance-value-range. X21 is the minimum value in the third impedance-value-range. k1 is an adjustment coefficient.

The equation indicates that a safe-voltage value is first set for the voltage value of the voltage output of the electrodes 301, to ensure that the electrodes 301 can output under extreme conditions, avoiding the case of no output which degrades the user experience. Meanwhile, the change of voltage value of the voltage output of the electrodes 301 has a positive correlation with the change of the impedance value, and a specific change of the voltage value depends on three points. The first is that a current impedance value and the minimum value in the third impedance-value-range determine a drop coefficient which is taken as a change trend of wearing condition compared with the normal wearing condition. The second is that the adjustment coefficient is set. The degree of compression has different changes according to different materials, sizes, and shapes of the electrodes 301 and even the type of the massager, therefore, different optimal change trends will occur. The adjustment coefficient k1 is set to 1 according to a recognized standard electrical stimulation massage device. The adjustment coefficient k1 is adjusted for other electrical stimulation massage devices according to comparative tests or user feedback to alleviate or aggravate the changing trend. The third is that an adjustable voltage value is set, a1+b1=V12, that is, the two limits of the adjustment range are the safe-voltage value a1 and the maximum output value V12, i.e., the voltage value of the present rank. The safe-voltage value a1 is in the range of 8V to 16V.

In some embodiments, the voltage output of the electrodes 301 is increased, in response to a determination that the impedance value decreases, when the impedance value is in the second impedance-value-range. In the entire second impedance-value-range, the voltage output of the electrodes 301 changes with the impedance value specifically as follows.

When the impedance value is in the second impedance-value-range, the voltage output of the electrodes satisfies an equation

$$V 21 = a 2 + b 2 ( \frac{X 22}{R 2} ) k 2 .$$

a2+b2=V22. V21 is a voltage value of the output electromagnetic pulse signal when the impedance value is in the second impedance-value-range. V22 is a voltage value of the present rank. a2 is a safe-voltage value. R2 is the impedance value in the second impedance-value-range. X22 is the maximum value of a third impedance-value-range. k2 is an adjustment coefficient.

The principle of the above equation is similar to that of the equation for the first impedance-value-range. A safe-voltage value is also set. Meanwhile, the change of the voltage value of the voltage output of the electrodes 301 has a negative correlation with the change of the impedance value, and a specific change of the voltage value depends on three points. The principles of b2 and k2 are similar to the aforementioned, but have a main difference that the adjustment is a negative-correlation change, that is, the maximum value of the third impedance-value-range and the current impedance value determine a drop coefficient which is taken as a change trend of wearing condition compared with the normal wearing condition. The safe-voltage value a2 is in the range of 8V to 16V.

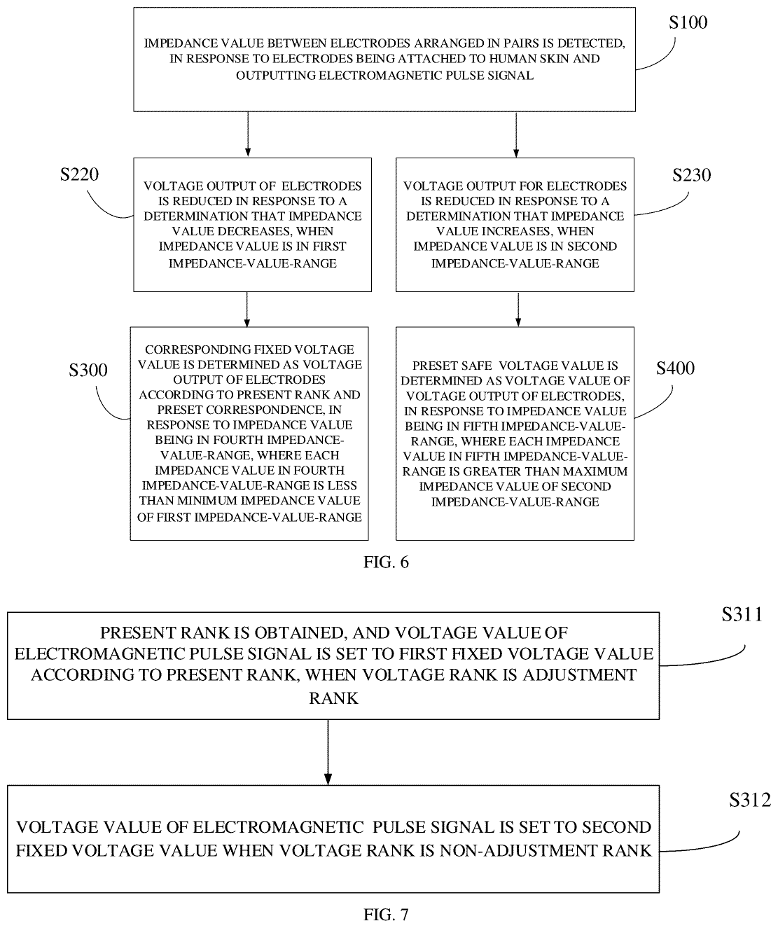

As illustrated in FIG. 6 and FIG. 7, the disclosure provides embodiments of a fourth impedance-value-range and a fifth impedance-value-range.

The control method includes the following.

At S300, a corresponding fixed voltage value is determined as the voltage output of the electrodes according to a present rank and a preset correspondence, in response to the impedance value being in a fourth impedance-value-range, where each impedance value in the fourth impedance-value-range is less than the minimum impedance value in the first impedance-value-range.

At S400, a preset safe-voltage value is determined as a voltage value of the voltage output of the electrodes, in response to the impedance value being in a fifth impedance-value-range, where each impedance value in the fifth impedance-value-range is greater than the maximum impedance value in the second impedance-value-range.

For the operation at S300 in the embodiment, the impedance value being in the fourth impedance-value-range indicates that the degree of adherence of skin is good. In this case, if the output voltage is not limited, the human body will bear a large current which is prone to cause discomfort. In this case, the electrical stimulation massage device will output a lower value corresponding to the present rank to ensure that the user has comfortable experience. The above-mentioned lower value may be obtained through preset settings. Corresponding associated data is set for different ranks and the voltage output of the electrodes 301. The corresponding fixed voltage is determined as the voltage output of the electrodes 301 value according to the present rank and the preset correspondence.

Another adjustment manner is provided as follows, where voltage ranks are set as adjustment ranks and non-adjustment ranks specifically as follows.

At S311, a present rank is obtained. A voltage value of the electromagnetic pulse signal is set to a first fixed voltage value according to the present rank, when the voltage rank is an adjustment rank.

At S312, a voltage value of the electromagnetic pulse signal is set to a second fixed voltage value when the voltage rank is a non-adjustment rank.

Each adjustment rank corresponds to a first fixed voltage value, and the first fixed voltage value is less than a voltage value of a voltage rank.

Specifically, since a voltage value corresponding to a low-voltage adjustment rank has little effect on the human body, at some low-voltage adjustment ranks working as the non-adjustment ranks, the voltage of the present rank can be directly output without voltage adjustment, that is, the second fixed voltage value is output. However, some high-voltage adjustment ranks are prone to causing tingling or uncomfortable massage when the impedance value is very small. Therefore, these high-voltage adjustment ranks may be subjected to voltage adjustment to be adjusted to a relatively small range and work as the adjustment rank.

Each first fixed voltage value corresponding to an adjustment rank needs to be pre-set. The voltage output of the electrodes 301 is directly set to a corresponding first fixed voltage value, in response to the impedance value being in a fourth impedance-value-range. The adjustment can be carried out slowly so that the user will not feel a sudden change. A rank with a voltage in the range of 8V to 16V can be considered as a non-adjustment rank, and the maximum value of the first fixed voltage value corresponding to the adjustment rank is also in the range of 8V to 16V.

For the operation at S400 in the embodiment, the impedance value being in the fifth impedance-value-range indicates that the degree of adherence of the skin with the electrodes 301 and the skin dryness condition turn into a bad condition which is no longer suitable for pulse output. The current will flow through a little part of the skin that is adhered to the electrode, and when the voltage is too large, tingling will be caused. In this case, it is recommended that the electrodes 301 output a safe rated low voltage to ensure that the user does not experience tingling.

In some embodiments, the safe rated low voltage can be set in the second impedance-value-range, and the voltage value of the output voltage for the electrodes 301 in the second impedance-value-range is the minimum. In this case, a preset safe-voltage value is determined as the voltage value of the voltage output of the electrodes 301, that is, the preset safe-voltage value can be the safe-voltage value a2. Of course, another voltage value less than a2 can be chosen as the preset safe-voltage value. The preset safe-voltage value is in the range of 8V to 16V.

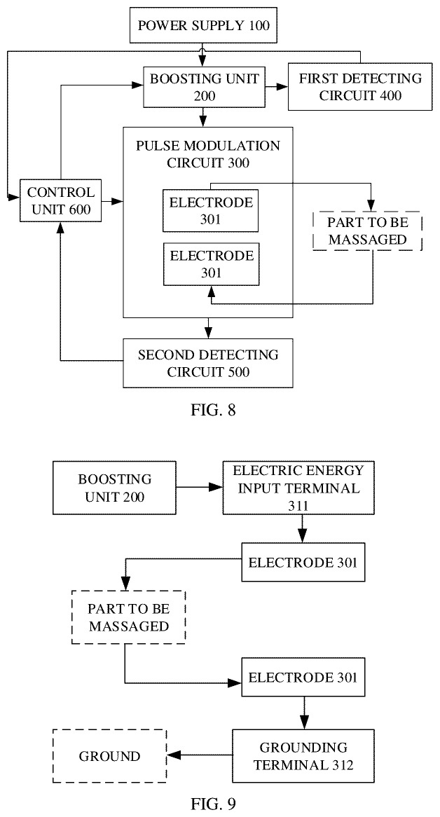

As illustrated in FIG. 8 to FIG. 15, the disclosure provides an embodiment of a control circuit of an electrical stimulation massage device.

The electrical stimulation massage device includes a power supply 100, a control unit 600, a boosting unit 200, electrodes 301 arranged in pairs, a pulse modulation circuit 300, a first detecting circuit 400, and a second detecting circuit 500. The boosting unit 200 is connected to the control unit 600 and the power supply 100 respectively. The boosting unit 200 is configured to boost an input voltage of the power supply 100 to a preset voltage under the control of the control unit 600, and output the preset voltage through a voltage output terminal of the boosting unit 200. The electrodes 301 is configured to be adhered to human skin at an area to be massaged. An electric energy input terminal 311 of the pulse modulation circuit 300 is connected to the voltage output terminal of the boosting unit 200. A first pulse transmitting terminal of the pulse modulation circuit 300 and a second pulse transmitting terminal of the pulse modulation circuit 300 each is connected to an electrode 301. A control terminal of the pulse modulation circuit 300 is connected to the control unit 600. The first detecting circuit 400 is connected to the control unit 600 and the voltage output terminal of the boosting unit 200 respectively, where the control unit 600 is configured to obtain an output voltage of the boosting unit 200 through the first detecting circuit 400. The second detecting circuit 500 is connected to the control unit 600. A sampling resistor R101 of the second detecting circuit 500 is connected in series between the pulse modulation circuit 300 and the ground. The control unit 600 is configured to obtain a sampling voltage of the sampling resistor R101 through the second detecting circuit 500. The control unit 600 is configured to obtain the impedance value between the electrodes 301 arranged in pairs according to the output voltage, resistance of the sampling resistor R101, and the sampling voltage of the sampling resistor R101.

Specifically, the boosting unit 200 includes a power input terminal, the voltage output terminal, and a control terminal. The pulse modulation circuit 300 includes the control terminal, the electric energy input terminal 311, a grounding terminal 312, the first pulse transmitting terminal, and the second pulse transmitting terminal. The first detecting circuit 400 includes a transmitting terminal 420 and a detecting terminal 410. The second detecting circuit 500 includes a transmitting terminal 520 and a detecting terminal 510. The electrodes 301 arranged in pairs include a first electrode and a second electrode.

In some embodiments, referring to FIG. 9, the boosting unit 200 is connected to the power supply 100 through the power input terminal. The power supply 100 supplies power for the boosting unit 200. The boosting unit 200 is further connected to the electric energy input terminal 311 of the pulse modulation circuit 300 through the voltage output terminal, and is configured to boost the input voltage of the power supply 100 to the preset voltage and transmit the preset voltage to the pulse modulation circuit 300 as a voltage value of an electromagnetic pulse signal. The boosting unit 200 is further connected to the control unit 600 through the control terminal, and performs a boosting process under the control of the control unit 600 to boost the input voltage of the power supply 100 to the preset voltage.

The pulse modulation circuit 300 is connected to the first electrode through the first pulse transmitting terminal, and is connected to the second electrode through the second pulse transmitting terminal. The pulse modulation circuit 300 is grounded through the grounding terminal 312 to form a current loop, which is equivalent to connecting to a negative pole of the power supply 100. The control terminal of the pulse modulation circuit 300 is connected to the control unit 600. The pulse modulation circuit 300 generates a pulse signal, i.e., the electromagnetic pulse signal, under the control of the control unit 600 based on the electric energy provided by the boosting unit 200. When the first electrode and the second electrode are turned on, the first pulse transmitting terminal outputs the electromagnetic pulse signal through the first electrode, and the second pulse transmitting terminal receives the electromagnetic pulse signal through the second electrode, and then the electromagnetic pulse signal is output through the grounding terminal 312 to form a pulse cycle. In this case, the first electrode and the second electrode are adhered to the part to be massaged to achieve an electrical conduction between the first electrode and the second electrode. The electromagnetic pulse signal is input to the part to be massaged through the electrodes 301, allowing users to experience electric stimulation and massage.

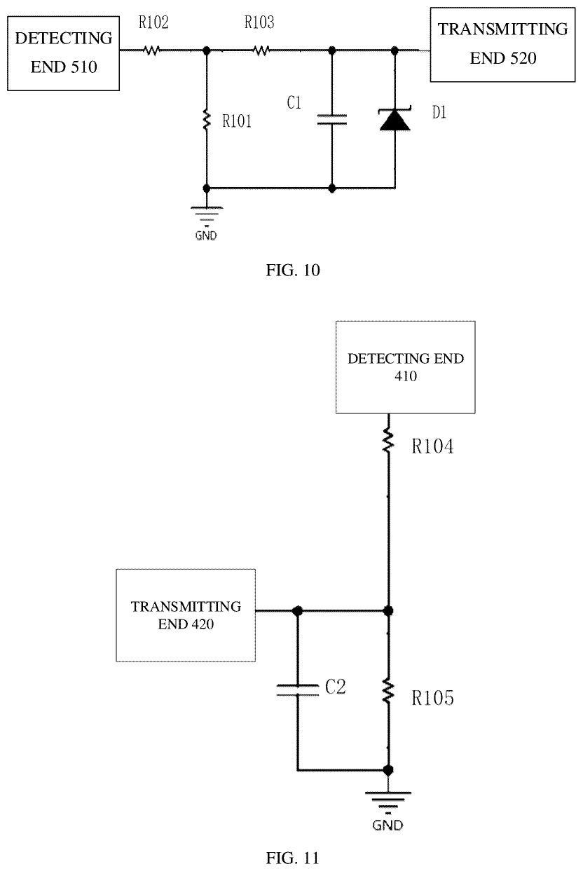

In some embodiments, referring to FIG. 10, the first detecting circuit 400 and the second detecting circuit 500 are both connected to the control unit 600 through their transmitting ends. The first detecting circuit 400 is connected to the voltage output terminal of the boosting unit 200 through the detecting terminal 410. The second detecting circuit 500 is connected in parallel to the sampling resistor R101 through the detecting terminal 510. In order to mitigate or even prevent the tingling at the part to be massaged caused by the excessive current of the electromagnetic pulse signal, and to allow the user to experience the entire electric stimulation massage without tingling, the control unit 600 first obtains a voltage value at the voltage output terminal of the boosting unit 200 through the first detecting circuit 400, that is, obtaining a specific voltage value of the boosted input voltage of the power supply 100, so as to determine whether the preset voltage reaches an expected value. The control unit 600 then obtains the sampling voltage of the sampling resistor R101 through the second detecting circuit 500. Finally, the control unit 600 obtains the output voltage of the boosting unit 200 and the sampling voltage of the sampling resistor R101, where the resistance of the sampling resistor R101 is stored. The control unit obtains, according to a preset algorithm, the impedance value between the electrodes 301 arranged in pairs, based on the output voltage, the resistance of the sampling resistor R101, and the sampling voltage of the sampling resistor R101. That is, a current flowing through the sampling resistor R101 is obtained through the resistance and the sampling voltage of the sampling resistor R101, and accordingly a current value of the electromagnetic pulse signal of the pulse modulation circuit 300 can be obtained. The voltage value of the electromagnetic pulse signal of the pulse modulation circuit 300 is obtained through the output voltage. A total resistance of the pulse modulation circuit 300 is obtained according to the current value and voltage value of the electromagnetic pulse signal, and the total resistance is taken as the impedance value between the electrodes 301 arranged in pairs. Alternatively, the impedance value between the electrodes 301 arranged in pairs is obtained by subtracting the resistance of the sampling resistor R101 from the total resistance. Alternatively, the impedance value between the electrodes 301 arranged in pairs is obtained by subtracting the resistance of the sampling resistor R101 and a preset error margin from the total resistance. The preset error margin can be internal resistance generated by wires or components of the pulse modulation circuit 300, internal resistance generated by the electrodes 301 due to its own material or shape, or internal resistance generated by other parts.

Furthermore, the control unit 600 obtains the impedance value of the part to be massaged in real-time through the above operations, and purposefully adjusts the output voltage of the boosting unit 200 according to the impedance value, thereby adjusting the current of the electromagnetic pulse signal of the pulse modulation circuit 300, so that the current value of the electromagnetic pulse signal for the part to be massaged can be kept in control during the massage, the part to be massaged is prevented from being subjected to strong electric stimulation, and painless massage is achieved.

In some embodiments, the control unit 600 includes a microcontroller unit (MUC) and peripheral circuits. The MCU is known as a single chip microcomputer or a single chip, which appropriately reduces the frequency and specifications of a central processing unit (CPU), and integrates a memory, a timer, a universal serial bus (USB), an analog-to-digital (A/D) converter, a universal asynchronous receiver/transmitter (UART), a programmable logic controller (PLC), a direct memory access (DMA), and other peripheral interfaces, and even a liquid crystal display (LCD) drive circuit on one chip to form a computer on chip to perform different combination controls for different applications. Each pin of the MUC is connected to a functional module, such as the boosting unit 200, the pulse modulation circuit 300, the first detecting circuit 400, and the second detecting circuit 500, to achieve the control and detection of the electric pulse. Of course, the control and detection of the electric pulse can be achieved by using a commonly used MCU on the market, and the performance requirements for the MCU are not high.

In some embodiments, the second detecting circuit 500 further includes a first protection resistor R103, a first capacitor C1, and a first Zener diode D1. The control unit 600 is connected between the pulse modulation circuit 300 and the sampling resistor R101 through the first protection resistor R103. The control unit 600 is further connected between the sampling resistor R101 and the ground through the first capacitor C1 and the first Zener diode D1 respectively. The anode of the first Zener diode D1 is grounded. Specifically, one terminal of the sampling resistor R101 is connected to the grounding terminal 312 of the pulse modulation circuit 300, and the other terminal of the sampling resistor R101 is connected to the ground. The current output from the pulse modulation circuit 300 flows through the sampling resistor R101. The voltage of the sampling resistor R101 is obtained by the control unit 600. By setting the first protection resistor R103 which is connected to the control unit 600 and the sampling resistor R101 respectively, the voltage input to the control unit 600 is prevented from being too large, and a voltage division processing is performed to effectively protect the control unit 600. By setting the first capacitor C1, a sampling signal is filtered to improve the accuracy of sampling data. By setting the first Zener diode D1, voltage stabilization is achieved. In some embodiments, the first Zener diode D1 may be a voltage regulator tube.

In some embodiments, three algorithm models for calculating the impedance value between electrodes 301 are provided.

Solution 1: The control unit 600 stores a first model for calculating the impedance value between the electrodes 301. The first model is

$$\{ \begin{matrix}{R}_{impedance} = \frac{{V}_{output}}{{I}_{modulation}} \\ {I}_{modulation} = \frac{{V}_{sampling}}{{R}_{sampling}}\end{matrix} ,$$

where R_{impedance} is an impedance value between the electrodes 301 arranged in pairs, V_{sampling} is a sampling voltage, R_{sampling} is resistance of the sampling resistor R101, Vₒᵤₜₚᵤₜ is an output voltage of the boosting unit 200, and I_{modulation} is a current of the pulse output by the pulse modulation circuit 300. First, a current flowing through the sampling resistor R101 is obtained through V_{sampling} and R_{sampling}, that is, the current I_{modulation} of the pulse output by the pulse modulation circuit 300 is obtained. The output voltage Vₒᵤₜₚᵤₜ of the boosting unit 200 is obtained. Resistance of the pulse modulation circuit 300 is obtained through Vₒᵤₜₚᵤₜ and I_{modulation}. The pulse output by the pulse modulation circuit 300 is the electromagnetic pulse signal. The resistance of the pulse modulation circuit 300 is an impedance value of the part to be massaged adhered to the electrodes 301 arranged in pairs.

Solution 2: The control unit 600 stores a second model for calculating the impedance value between the electrodes 301. The second model is

$$\{ \begin{matrix}{R}_{impedance} + {R}_{sampling} = \frac{{V}_{output}}{{I}_{modulation}} \\ {I}_{modulation} = \frac{{V}_{sampling}}{{R}_{sampling}}\end{matrix} ,$$

where R_{impedance} is an impedance value between the electrodes 301 arranged in pairs, V_{sampling} is a sampling voltage, R_{sampling} is resistance of the sampling resistor R101, Vₒᵤₜₚᵤₜ is an output voltage of the boosting unit 200, and I_{modulation} is a current of the pulse output by the pulse modulation circuit 300. Compared with the solution 1, resistance of the pulse modulation circuit 300 includes not only the impedance value of the part to be massaged adhered to the electrodes 301 arranged in pairs, but also the resistance R_{sampling} of the sampling resistor R101, which is mainly because the resistance of the sampling resistor R101 is relatively large in the disclosure. The difference between the impedance value of the part to be massaged and the resistance of the sampling resistor R101 is not very large. The resistance of the sampling resistor R101 cannot be ignored. In this way, the resistance of the sampling resistor R101 is included to improve accuracy.

Solution 3: The control unit 600 stores a third model for calculating the impedance value between the electrodes 301. The third model is

$$\{ \begin{matrix}{R}_{impedance} + {R}_{sampling} + {R}_{margin} = \frac{{V}_{output}}{{I}_{modulation}} \\ {I}_{modulation} = \frac{{V}_{sampling}}{{R}_{sampling}}\end{matrix} ,$$

where R_{impedance} is an impedance value between the electrodes 301 arranged in pairs, V_{sampling} is a sampling voltage, R_{sampling} is resistance of the sampling resistor R101, Vₒᵤₜₚᵤₜ is an output voltage of the boosting unit 200, I_{modulation} is a current of the pulse output by the pulse modulation circuit 300, and R_{margin} in is a preset error margin. Compared with the solution 2, the preset error margin is added. The preset error margin can be the internal resistance generated by the wires or components of the pulse modulation circuit 300, the internal resistance generated by the electrodes 301 due to its own material or shape, or the internal resistance generated by other parts. The preset error margin can be calculated by experiment or obtained by theoretical calculation. The preset error margin is added to further improve the accuracy.

The second detecting circuit 500 further includes a second protection resistor R102. The second protection resistor R102 is connected in series between the pulse modulation circuit 300 and the sampling resistor R101. The second protection resistor R102 is set to reduce the electric energy flowing into the control unit 600, or reduce the voltage input to the control unit 600 through voltage division, thereby protecting the entire second detecting circuit 500.

In some embodiments, referring to FIG. 11, the first detecting circuit 400 includes a first voltage-dividing resistor R104 and a second voltage-dividing resistor R105. The first voltage-dividing resistor R104 is respectively connected to the voltage output terminal of the boosting unit 200 and the second voltage-dividing resistor R105. The other terminal of the second voltage-dividing resistor R105 is grounded. The control unit 600 is connected to a connection node between the first voltage-dividing resistor R104 and the second voltage-dividing resistor R105 to obtain a divided voltage of the second voltage-dividing resistor R105. The control unit 600 obtains the output voltage of the boosting unit 200 according to the divided voltage of the second voltage-dividing resistor R105, resistance of the first voltage-dividing resistor R104, and resistance of the second voltage-dividing resistor R105.

Specifically, the first voltage-dividing resistor R104 and the second voltage-dividing resistor R105 obtain the output voltage of the boosting unit 200, based on the divided voltage of the first voltage-dividing resistor R104 and the divided voltage of the second voltage-dividing resistor R105. Then the resistance of the second voltage-dividing resistor R105 is reduced, so that the control unit can directly obtain a voltage of the second voltage-dividing resistor R105 without the need for protection or shunting from additional components. The resistance of the first voltage-dividing resistor R104 may be much larger than the resistance of the second voltage-dividing resistor R105. The voltage value of the second voltage-dividing resistor R105 is reduced. The control unit 600 can directly obtain the output voltage of the boosting unit 200, based on the known resistance of the first voltage-dividing resistor R104, the known resistance of the second voltage-dividing resistor R105, and the divided voltage of the second voltage-dividing resistor R105. Determination of the resistance of the first voltage-dividing resistor R104 and the resistance of the second voltage-dividing resistor R105 depends on two aspects: first, a range of the output voltage of the boosting unit 200 needs to be considered; second, a voltage limit of a value-determining terminal of the control unit 600 needs to be considered. A resistance ratio of the second voltage-dividing resistor R105 to the first voltage-dividing resistor R104 ranges from 1:37 to 1:72. The resistance of the second voltage-dividing resistor R105 is, for example, 10kΩ and can be in a value range of 9kΩ to 11kΩ. The resistance of the first voltage-dividing resistor R104 is, for example, 510kΩ and can be in a value range of 450kΩ to 570kΩ.

In some embodiments, the pulse modulation circuit 300 further includes at least one group of control arms. Each control arm includes a first control switch 321 and a second control switch 324. The control unit 600 is respectively connected to a control terminal of the first control switch 321 and a control terminal of the second control switch 324 to respectively control on and off of the first control switch 321 and the second control switch 324. An input terminal of the first control switch 321 is connected to the electric energy input terminal 311. An output terminal of the second control switch 324 is connected to the ground. An output terminal of the first control switch 321 is connected to one of the first pulse transmitting terminal and the second pulse transmitting terminal. An input terminal of the second control switch 324 is connected to the other one of the first pulse transmitting terminal and the second pulse transmitting terminal.

Specifically, when a boosting circuit stably inputs an input voltage and the electrodes 301 are adhered to the part to be massaged, the control unit 600 controls the on and off of the first control switch 321 and the second control switch 324 to generate a pulse signal, i.e., an electromagnetic pulse signal. The electric energy input by the boosting circuit is sequentially output through the first control switch 321, the first electrode, the part to be massaged, the second electrode, and the second control switch 324, and then flows through the sampling resistor R101 of the second detecting circuit 500. The part to be massaged is stimulated through a pulse current, so that the part to be massaged experiences the feeling of massage. The current flowing through the part to be massaged is adjusted by adjusting the input voltage, to achieve different massage strengths, and achieve different massage techniques in combination with different pulse frequencies. The control unit 600 is connected to the first control switch 321 through a control terminal 331, and is connected to the second control switch 324 through a control terminal 334.

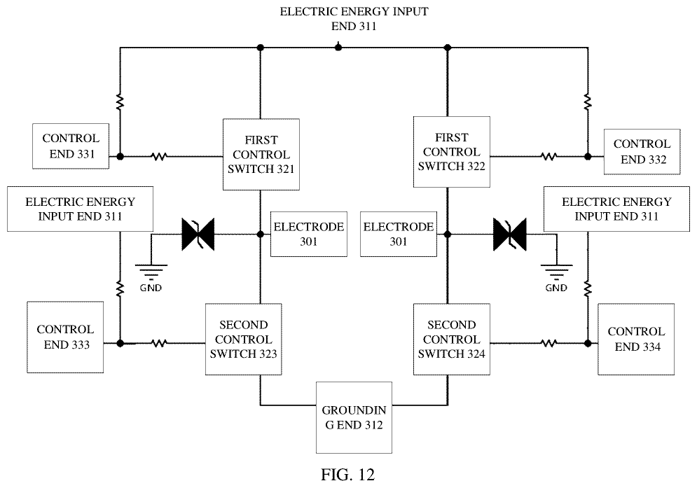

In some embodiments, referring to FIG. 12, two groups of control arms are provided. Output ends of two first control switches (321, 322) are respectively connected to the first pulse transmitting terminal and the second pulse transmitting terminal. Input ends of two second control switches (323, 324) are respectively connected to the first pulse transmitting terminal and the second pulse transmitting terminal. An H-bridge circuit is formed by the four control switches to realize rapid control of alternate on and off of the two groups of control arms. The control unit 600 is connected to a first control switch 322 through a control terminal 332, and is connected to a second control switch 323 through a control terminal 333.

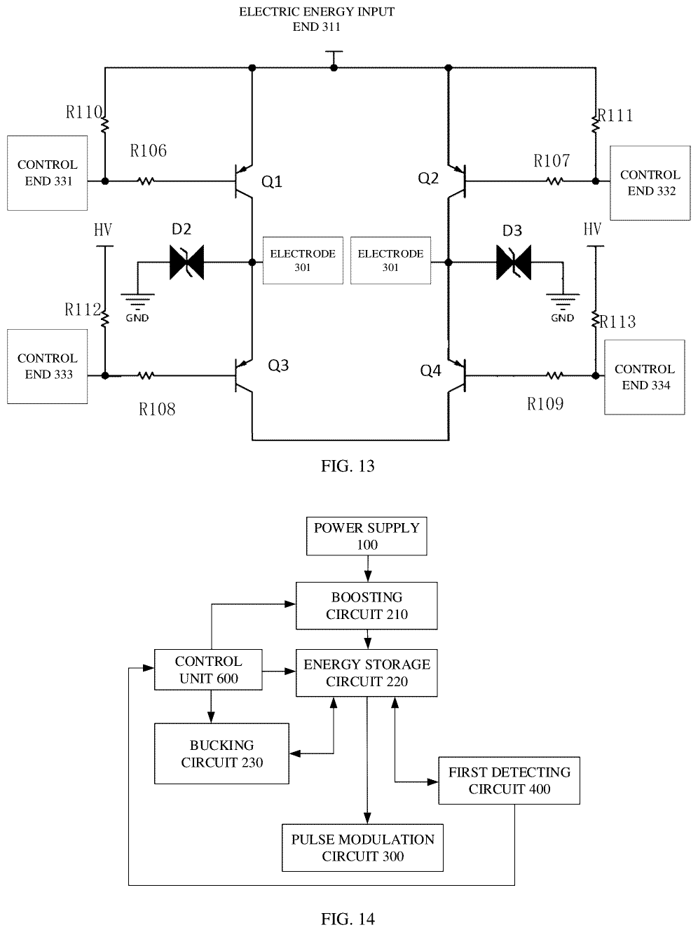

In some embodiments, referring to FIG. 13, the first control switches (321, 322) and the second control switches (323, 324) are all triodes. The H-bridge circuit is taken as an example. Two of the triodes form a group of control arms. A first triode Q1, a second triode Q2, a third triode Q3, and a fourth triode Q4 are included. The first triode Q1 and the second triode Q2 serve as the first control switches (321, 322). The third triode Q3 and the fourth triode Q4 serve as the second control switches (323, 324). An emitter of the first triode Q1 and an emitter of the second triode Q2 are both connected to a power input terminal of the boosting unit 200, serving as the electric energy input terminal 311 of the pulse modulation circuit 300. A base of the first triode Q1 and a base of the second triode Q2 are both connected to a control terminal of the control unit 600. Collectors of the first triode Q1 and the second triode Q2 are respectively connected to the two electrodes 301. An emitter of the third triode Q3 and an emitter of the fourth triode Q4 are connected to the two electrodes 301 respectively. Collectors of the third triode Q3 and the fourth triode Q4 are connected to the grounding terminal 312 of the pulse modulation circuit 300. Abase of the third triode Q3 and a base of the fourth triode Q4 are connected to the control terminal of the control unit 600. The control unit 600 can control on and off of the first triode Q1, the second triode Q2, the third triode Q3, and the fourth triode Q4 respectively. In some embodiments, the control unit 600 can control on and off of the first triode Q1 and the fourth triode Q4 at the same time, and control on and off of the second triode Q2 and the third triode Q3 at the same time.

More specifically, the power input terminal of the boosting unit 200 is connected to the control unit 600 through a pull-up resistor to provide a voltage for driving on and off the triodes. Resistors are connected in series between the control unit 600 and the bases of the triodes to protect the control unit 600 and generate a driving voltage at the bases to achieve conduction of the triodes. The first pulse transmitting terminal and the second pulse transmitting terminal each are grounded through a bidirectional variable resistance diode (D2, D3) to achieve bidirectional blocking between the electrodes and the ground, so that the current can flow back to the ground. The electric energy input terminal 311 of the pulse modulation circuit 300 is connected to the control unit 600 through a resistor R110 and the control terminal 331. The electric energy input terminal 311 of the pulse modulation circuit 300 is connected to the control unit 600 through a resistor R111 and the control terminal 332. The electric energy input terminal 311 of the pulse modulation circuit 300 is connected to the control unit 600 through a resistor R112 and the control terminal 333. The electric energy input terminal 311 of the pulse modulation circuit 300 is connected to the control unit 600 through a resistor R113 and the control terminal 334. Moreover, a resistor R106 is connected in series between the control terminal 331 and the base of the first triode Q1. A resistor R107 is connected in series between the control terminal 332 and the base of the second triode Q2. A resistor R108 is connected in series between the control terminal 333 and the base of the third triode Q3. A resistor R109 is connected in series between the control terminal 334 and the base of the fourth triode Q4.

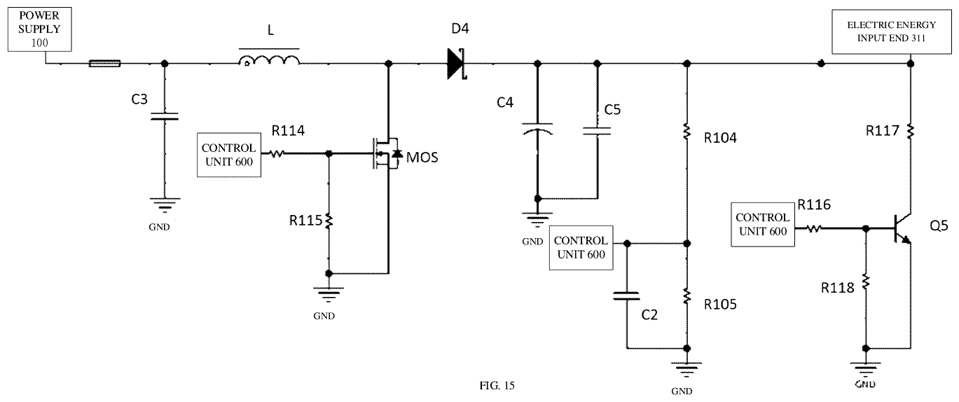

In some embodiments, referring to FIG. 14 and FIG. 15, the boosting unit 200 includes a power input terminal connected to the power supply 100, a boosting circuit 210, an energy storage circuit 220, a bucking circuit 230, and a voltage output terminal 201 connected to the pulse modulation circuit 300. An input terminal of the boosting circuit 210 is connected to the power input terminal. A control terminal of the boosting circuit 210 is connected to the control unit 600. An input terminal of the energy storage circuit 220 is connected to a voltage output terminal of the boosting circuit 210. An output terminal of the energy storage circuit 220 is connected to the voltage output terminal 201. A control terminal of the bucking circuit 230 is connected to the control unit 600. An input terminal of the bucking circuit 230 is connected to the voltage output terminal 201. The control unit 600 is configured to control, according to the preset voltage and the impedance value between the electrodes arranged in pairs, the boosting circuit and/or the energy storage circuit to perform a boosting process, and/or the bucking circuit to perform a bucking process, so as to control the voltage output terminal to output the preset voltage to the pulse modulation circuit.

Specifically, the power input terminal of the boosting circuit 210 is connected to the power input terminal to obtain a voltage of the power supply 100. The control terminal of the boosting circuit 210 is connected to the control unit 600 to receive a control instruction and boost the voltage of the power supply 100. The input terminal of the energy storage circuit 220 is connected to the voltage output terminal of the boosting circuit 210 to store energy of the boosted voltage. The output terminal of the energy storage circuit 220 is connected to the voltage output terminal 201 to output the preset voltage to the voltage output terminal 201. The control terminal of the bucking circuit 230 is connected to the control unit 600. The input terminal of the bucking circuit 230 is connected to the voltage output terminal 201 to reduce an output voltage of the boosting circuit 210 to the voltage output terminal 201 according to the control instruction.

When the impedance value between the electrodes 301 arranged in pairs increases, the control unit 600 controls the bucking circuit 230 to perform a bucking process on the output voltage at the voltage output terminal 201. When the impedance value between the electrodes 301 arranged in pairs decreases, the control unit 600 controls the voltage boosting circuit 210 to boost the voltage output by the power supply 100, so as to dynamically maintain an output power of the voltage output terminal 201 to be unchanged.

The boosting circuit 210 includes an inductor L and a metal oxide semiconductor field effect transistor (MOS). One terminal of the inductor L is connected to the power input terminal of the boosting circuit 210, and the other terminal of the inductor L is connected to the voltage output terminal of the boosting circuit 210. The gate of the MOS is connected to the control terminal of the boosting circuit 210. The drain of the MOS is connected between the inductor L and the voltage output terminal of the boosting circuit 210. The source of the MOS is grounded. The MOS is mainly configured as a current on-off switch. After the gate of the MOS is connected to the control terminal of the boosting circuit 210, the MOS can receive a control instruction from the control unit 600, and is turned on or off according to the control instruction of the control unit 600. When the MOS is turned on, a current of the inductor L flows to the ground through the MOS, so that the power supply 100 charges the inductor L. When the MOS is turned off, the current of the inductor L flows to the energy storage circuit 220, so as to boost the voltage output by the power supply 100. A capacitor C3 is connected between the power supply 100 and the inductor L, and is grounded to achieve filtering.

A resistor R114 is connected in series between the gate of the MOS and the control terminal of the boosting circuit 210, to protect the control unit 600. Moreover, the gate of the MOS is further connected to a resistor R115 and is grounded, to ensure that the control unit 600 is grounded without load and to prevent the MOS from being turned on.

A voltage output circuit further includes a diode D4 connected in series between the voltage output terminal of the boosting circuit 210 and the input terminal of the energy storage circuit 220; and/or, the energy storage circuit 220 is a capacitive energy storage circuit 220. When the MOS of the boosting circuit 210 is turned off, the current of the inductor L flows to the energy storage circuit 220 through the diode D4, so that a voltage output from the energy storage circuit 220 to the voltage output terminal 201 is the sum of a voltage output by the inductor L and an energy storage voltage of the energy storage circuit 220, and voltage boosting is therefore achieved.

The energy storage circuit 220 is the capacitive energy storage circuit 220 which includes a fourth capacitor C4 and a fifth capacitor C5 connected in parallel between the input terminal and the output terminal of the energy storage circuit 220. The other ends of the fourth capacitor C4 and the fifth capacitor C5 are grounded. The fourth capacitor C4 and the fifth capacitor C5 are mainly configured to store energy. The voltage output from the energy storage circuit 220 to the voltage output terminal 201 is the sum of the voltage output by the inductor L, the voltage of the fourth capacitor C4, and the voltage of the fifth capacitor C5, so that voltage boosting is achieved. Energy storage capacity of the fourth capacitor C4 is greater than energy storage capacity of the fifth capacitor C5.

The bucking circuit 230 includes a first resistor R116, a fifth triode Q5, a second resistor R117, and a third resistor R18. The first resistor R116 is connected in series between the control terminal of the bucking circuit 230 and the base of the fifth triode Q5. The emitter of the fifth triode Q5 is grounded. One terminal of the third resistor R18 is connected between the first resistor R116 and the base of the fifth triode Q5, and the other terminal of the third resistor R18 is grounded. The second resistor R117 is connected in series between the input terminal of the bucking circuit 230 and the collector of the fifth triode Q5. Specifically, when the voltage output from the energy storage circuit 220 to the voltage output terminal 201 is larger than the preset voltage, the control unit 600 controls the triode Q5 to be turned on, and the bucking circuit 230 performs bucking process on the voltage of the inductor L and the energy storage circuit 220, so that the voltage output from the energy storage circuit 220 to the voltage output terminal 201 is reduced to the preset voltage, and the preset voltage is outputted to the pulse modulation circuit 300 through the voltage output terminal 201.

In the disclosure, an electrical stimulation massage device is provided and includes a memory and a processor. A computer program is stored in the memory. The computer program, when executed by the processor, is operable with the processor to implement a control method for the electrical stimulation massage device.

In the disclosure, a non-transitory computer-readable storage medium storing a computer program is provided. The computer program, when executed by a processor, is operable with the processor to implement a control method for the electrical stimulation massage device.

The above descriptions are only some embodiments of the disclosure and are not intended to limit the scope of the disclosure. All equivalent changes or modifications made according to the scope of the patent application of the disclosure are covered by the disclosure.

What is claimed is:

1. A control method for an electrical stimulation massage device, wherein
the electrical stimulation massage device comprises electrodes arranged in pairs, the electrodes are configured to be adhered to human skin at an area to be massaged, and the control method comprises:
detecting an impedance value between the electrodes arranged in pairs, in response to the electrodes being adhered to the human skin and outputting an electromagnetic pulse signal;
reducing a voltage output of the electrodes, in response to a determination that the impedance value decreases, when the impedance value is in a first impedance-value-range; and
reducing the voltage output of the electrodes, in response to a determination that the impedance value increases, when the impedance value is in a second impedance-value-range,
wherein the maximum value of the first impedance-value-range is less than or equal to the minimum value of the second impedance-value-range.

2. The control method of claim 1, further comprising:
adjusting the voltage output of the electrodes to be less than a voltage output corresponding to a present rank, in response to the impedance value being in the first impedance-value-range or the second impedance-value-range.

3. The control method of claim 1, further comprising:
outputting the voltage output corresponding to a present rank to the electrodes, in response to the impedance value being in a third impedance-value-range, wherein
the third impedance-value-range is between the first impedance-value-range and the second impedance-value-range.

4. The control method of claim 3, further comprising:
controlling the voltage output of the electrodes to be gradually recovered to the voltage output corresponding to the present rank, in response to a determination that the impedance value changes from the first impedance-value-range or the second impedance-value-range to the third impedance-value-range.

5. The control method of claim 3, further comprising:
controlling the voltage output of the electrodes by performing a bucking process according to the voltage output corresponding to the present rank, in response to a determination that the impedance value changes from the third impedance-value-range to the first impedance-value-range or the second impedance-value-range.

6. The control method of claim 1, further comprising:
increasing the voltage output to the electrodes, in response to a determination that the impedance value increases, when the impedance value is in the first impedance-value-range.

7. The control method of claim 3, wherein when the impedance value is in the first impedance-value-range, the voltage output of the electrodes satisfies an equation
$$V 11 = a 1 + b 1 ( \frac{R 1}{X 21} ) k 1 ,$$
$$wherein$$
$$a 1 + b 1 = V 12 ;$$
V11 is a voltage value of the voltage output of the electrodes when the impedance value is in the first impedance-value-range;
V12 is a voltage value of the present rank;
a1 is a safe-voltage value;
R1 is the impedance value in the first impedance-value-range;
X21 is the minimum value in the third impedance-value-range; and
k1 is an adjustment coefficient.

8. The control method of claim 1, further comprising:
determining a corresponding fixed voltage value as the voltage output of the electrodes according to a present rank and a preset correspondence, in response to the impedance value being in a fourth impedance-value-range, wherein each impedance value in the fourth impedance-value-range is less than the minimum impedance value in the first impedance-value-range.

9. The control method of claim 1, further comprising:
setting voltage ranks as adjustment ranks and non-adjustment ranks, in response to the impedance value being in a fourth impedance-value-range, wherein the impedance value is less than the minimum impedance value in the first impedance-value-range;
obtaining a present rank;
setting a voltage value of the electromagnetic pulse signal to a first fixed voltage value according to the present rank, when the voltage rank is an adjustment rank; and
setting a voltage value of the electromagnetic pulse signal to a second fixed voltage value when the voltage rank is a non-adjustment rank, wherein
each adjustment rank corresponds to a first fixed voltage value, and the first fixed voltage value is less than a voltage value of a voltage rank.

10. The control method of claim 1, comprising:
increasing the voltage output of the electrodes, in response to a determination that the impedance value decreases, when the impedance value is in the second impedance-value-range.

11. The control method of claim 3, wherein when the impedance value is in the second impedance-value-range, the voltage output of the electrodes satisfies an equation
$$V 21 = a 2 + b 2 ( \frac{X 22}{R 2} ) k 2 ,$$
$$wherein$$
$$a 2 + b 2 = V 22 ;$$
V21 is a voltage value of the output electromagnetic pulse signal when the impedance value is in the second impedance-value-range;
V22 is a voltage value of a present rank;
a2 is a safe-voltage value;
R2 is the impedance value in the second impedance-value-range;
X22 is the maximum value of a third impedance-value-range; and
k2 is an adjustment coefficient.

12. The control method of claim 1, further comprising:
determining a preset safe-voltage value as a voltage value of the voltage output of the electrodes, in response to the impedance value being in a fifth impedance-value-range, wherein each impedance value in the fifth impedance-value-range is greater than the maximum impedance value in the second impedance-value-range.

13. The control method of claim 1, wherein detecting the impedance value between the electrodes arranged in pairs comprises:
obtaining a current value corresponding to the electromagnetic pulse signal, and obtaining a total impedance value of the electrodes according to the current value; and
taking the total impedance value as the impedance value.

14. The control method of claim 1, wherein detecting the impedance value between the electrodes arranged in pairs comprises:
obtaining a current value corresponding to the electromagnetic pulse signal, and obtaining a total impedance value of the electrodes according to the current value; and
taking the total impedance value as the impedance value after deducting internal resistance of an internal component from the total impedance value.

15. An electrical stimulation massage device, comprising:
a power supply and a control unit;
a boosting unit connected to the control unit and the power supply respectively, wherein the boosting unit is configured to boost an input voltage of the power supply to a preset voltage under the control of the control unit, and output the preset voltage through a voltage output terminal of the boosting unit;
electrodes configured to be adhered to human skin at an area to be massaged;
a pulse modulation circuit, wherein an electric energy input terminal of the pulse modulation circuit is connected to the voltage output terminal of the boosting unit, a first pulse transmitting terminal of the pulse modulation circuit and a second pulse transmitting terminal of the pulse modulation circuit each is connected to an electrode, and a control terminal of the pulse modulation circuit is connected to the control unit;
a first detecting circuit connected to the control unit and the voltage output terminal of the boosting unit respectively, wherein the control unit is configured to obtain an output voltage of the boosting unit through the first detecting circuit; and
a second detecting circuit connected to the control unit, wherein a sampling resistor of the second detecting circuit is connected in series between the pulse modulation circuit and a ground, and the control unit is configured to obtain a sampling voltage of the sampling resistor through the second detecting circuit, wherein
the control unit is configured to: detect an impedance value between the electrodes arranged in pairs, in response to the electrodes being adhered to human skin and outputting an electromagnetic pulse signal; reduce a voltage output to the electrodes in response to a determination that the impedance value decreases, when the impedance value is in a first impedance-value-range; and reduce the voltage output of the electrodes in response to a determination that the impedance value increases, when the impedance value is in a second impedance-value-range, wherein the maximum value of the first impedance-value-range is less than or equal to the minimum value of the second impedance-value-range, and a voltage value of the electromagnetic pulse signal is controlled.

16. The electrical stimulation massage device of claim 15, wherein in terms of detecting the impedance value between the electrodes arranged in pairs, the control unit is configured to:
detect the output voltage at the voltage output terminal of the boosting unit through the first detecting circuit;
detect the sampling voltage of the sampling resistor through the second detecting circuit; and
obtain the impedance value between the electrodes arranged in pairs according to the output voltage of the boosting unit, resistance of the sampling resistor, and the sampling voltage of the sampling resistor.

17. The electrical stimulation massage device of claim 16, wherein in terms of reducing the voltage output of the electrodes comprises:
reduce the output voltage at the voltage output terminal of the boosting unit.

18. The electrical stimulation massage device of claim 15, wherein the pulse modulation circuit further comprises:
at least one group of control arms, wherein each control arm comprises a first control switch and a second control switch, the control unit is respectively connected to a control terminal of the first control switch and a control terminal of the second control switch to respectively control on and off of the first control switch and the second control switch, an input terminal of the first control switch is connected to the electric energy input terminal, an output terminal of the second control switch is connected to the ground, an output terminal of the first control switch is connected to one of the first pulse transmitting terminal and the second pulse transmitting terminal, and an input terminal of the second control switch is connected to the other one of the first pulse transmitting terminal and the second pulse transmitting terminal.

19. The electrical stimulation massage device of claim 15, wherein the boosting unit comprises a power input terminal connected to the power supply, a boosting circuit, an energy storage circuit, and a bucking circuit, and the voltage output terminal connected to the pulse modulation circuit, wherein an input terminal of the boosting circuit is connected to the power input terminal, and a control terminal of the boosting circuit is connected to the control unit; an input terminal of the energy storage circuit is connected to a voltage output terminal of the boosting circuit, and an output terminal of the energy storage circuit is connected to the voltage output terminal; a control terminal of the bucking circuit is connected to the control unit, and an input terminal of the bucking circuit is connected to the voltage output terminal, wherein the control unit is configured to control, according to the preset voltage and the impedance value between the electrodes arranged in pairs, the boosting circuit and/or the energy storage circuit to perform a boosting process, and/or the bucking circuit to perform a bucking process, so as to control the voltage output terminal to output the preset voltage to the pulse modulation circuit.

20. A non-transitory computer-readable storage medium storing a computer program, wherein the computer program, when executed by a processor of an electrical stimulation massage device, is operable with the processor to:
detect an impedance value between electrodes arranged in pairs of the electrical stimulation massage device, in response to the electrodes being adhered to the human skin and outputting an electromagnetic pulse signal;
reduce a voltage output to the electrodes, in response to a determination that the impedance value decreases, when the impedance value is in a first impedance-value-range; and
reduce the voltage output of the electrodes, in response to a determination that the impedance value increases, when the impedance value is in a second impedance-value-range, wherein
the maximum value of the first impedance-value-range is less than or equal to the minimum value of the second impedance-value-range.
